(19) United States
(12) Patent Application Publication
(10) Pub. No.: US 2020/0321130 A1
(43) Pub. Date: Oct. 8, 2020

(54) SYSTEM, METHOD AND COMPUTER-ACCESSIBLE MEDIUM FOR DETERMINING BREAST CANCER RISK

(71) Applicants: THE TRUSTEES OF COLUMBIA UNIVERSITY IN THE CITY OF NEW YORK, NEW YORK, NY (US); UNIVERSITY OF CALIFORNIA SAN FRANCISCO, SAN FRANCISCO, CA (US)

(72) Inventors: RICHARD HA, White Plains, NY (US); PETER CHANG, San Francisco, CA (US)

(21) Appl. No.: 16/763,389

(22) PCT Filed: Nov. 12, 2018
(86) PCT No.: PCT/US18/60271

§ 371 Date: May 12, 2020

Related U.S. Application Data
(60) Provisional application No. 62/585,452, filed on Nov. 13, 2017.

(51) Int. Cl. G16H 50/30; G06T 3/40; G06T 7/00; G06N 3/08; G16H 30/40; G06N 3/04

(52) U.S. Cl. CPC G16H 50/30; G06T 3/40; G06T 7/0012; G06T 2207/30068; G16H 30/40; G06N 3/04; G06T 2207/20084; G06N 3/084

(57) ABSTRACT

An exemplary system, method and computer-accessible medium for determining a risk of developing breast cancer for a patient(s) can include, for example receiving an image(s) of an internal portion(s) of a breast of the patient(s), and determining the risk by applying a neural network(s) to the image(s). The neural network can be a convolutional neural network (CNN). The CNN can include a plurality of layers. Each of the layers can have a different number of feature channels. The CNN can include at least four layers. A first layer of the at least four layers can have 256×256×16 feature channels, a second layer of the at least four layers can have 128×128×32 feature channels, a third layer of the at least four layers can have 64×64×64 feature channels, and a fourth layer of the at least four layers can have 32×32×128 feature channels.

### CROSS-REFERENCE TO RELATED APPLICATION(S)

This application relates to and claims priority from U.S. Patent Application No. 62/585,452, filed on Nov. 13, 2017, the entire disclosure of which is incorporated herein by reference.

### FIELD OF THE DISCLOSURE

The present disclosure relates generally to breast cancer, and more specifically, to exemplary embodiments of exemplary system, method, and computer-accessible medium for determining breast cancer risk.

### BACKGROUND INFORMATION

Breast cancer is a leading cause of death worldwide and is the second most common cause of cancer deaths among women in the United States. (See, e.g., Reference 1). One in eight women can develop breast cancer; however the risk is not homogeneously distributed throughout the population. While some risk factors have been established, the majority of women diagnosed with breast cancer have no identifiable risk. (See, e.g., Reference 2). This can limit the ability of the medical community to determine high risk versus low risk women.

Evidence for stratifying the risk of developing breast cancer lies in mammographic breast density, defined as the proportion of radiopaque epithelial and stromal tissue compared to radiolucent fat. (See, e.g., Reference 3). In 1976, breast density as a cancer risk factor was determined, with four distinct classifications based on parenchymal patterns: primarily fat (“N1”), ductal prominence involving up to one-fourth of the breast (“P1”), ductal prominence involving more than one-fourth of the breast (“P2”), and severe ductal prominence (“DY”). (See, e.g., Reference 4).

Further studies have described more quantitative categorization of breast density as it relates to cancer predisposition, such as the Tabar classification. (See, e.g., References 5 and 6). The American College of Radiology Breast Imaging Reporting and Data System (“BI-RADS”) defines four categories: (i) entirely fatty, (ii) scattered fibroglandular densities, (iii) heterogeneously dense, and (iv) extremely dense. Several studies have examined the correlation of breast cancer risk and BI-RADS breast density criteria.

A large prospective study (see, e.g., Reference 7) showed risk increase with a higher BI-RADS category, with heterogeneously dense breasts (“BI-RADS 3”) 2.8 and extremely dense breasts (“BI-RADS 4”) being 4.0 times more likely to develop cancer compared to entirely fatty breasts (“BI-RADS 1”). (See, e.g., Reference 7). Similarly, an increase in BI-RADS breast density has been shown to correlate with an increased risk of breast cancer over a 3 year follow up. (See, e.g., Reference 8). Beyond the correlation of breast density and cancer risk, evidence has shown increased density to be an independent risk factor beyond a masking effect, as it represents the amount of stromal and epithelial tissue from which breast cancer derives. (See, e.g., Reference 3).

The current climate of changing breast cancer screening recommendations by the United States Preventive Services Task Force and American Cancer Society has demonstrated a consistent trend toward later, less frequent, screening, unless a woman is considered to be high risk. This makes the challenge of defining the high risk group within the general population even more important. (See, e.g., References 9 and 10). According to the Breast Cancer Surveillance Consortium database, almost half (e.g., 47%) of the population falls into the category of dense breasts (e.g., Bi-RADS 3 and 4) and therefore can be classified as high risk. (See, e.g., Reference 11). Thus, a more individualized stratification is needed to appropriately predict breast cancer risk and therefore designate the most appropriate screening regimen. While advances in imaging technology have provided high quality mammograms with increased clarity, the question remains: is there something beyond the amount of breast density that is not appreciated by the human eye that may affect risk?

Thus, it may be beneficial to provide exemplary system, method, and computer-accessible medium for determining breast cancer risk, which can address and/or overcome at least some of the deficiencies described herein above.

### SUMMARY OF EXEMPLARY EMBODIMENTS

An exemplary system, method and computer-accessible medium for determining a risk of developing breast cancer for a patient(s) can include, for example receiving an image(s) of an internal portion(s) of a breast of the patient(s), and determining the risk by applying a neural network(s) to the image(s). The neural network can be a convolutional neural network (CNN). The CNN can include a plurality of layers. Each of the layers can have a different number of feature channels. The CNN can include at least four layers. A first layer of the at least four layers can have 256×256×16 feature channels, a second layer of the at least four layers can have 128×128×32 feature channels, a third layer of the at least four layers can have 64×64×64 feature channels, and a fourth layer of the at least four layers can have 32×32×128 feature channels.

In some exemplary embodiments of the present disclosure, the CNN can include 3×3 convolutional kernels. Overfitting of the risk can be prevented using the 3×3 convolutional kernels. The CNN can exclude pooling layers. The image can be downsampled using, for example, a 3×3 convolutional kernel. The 3×3 convolutional kernel can have a stride length of 2. The risk can be determined by modeling non-linear functions using a rectified linear unit to limit drift of layer activations. The batch normalization can be performed between the ReLu layer(s) and a convolutional layer. The CNN can include four strided convolutions. The risk(s) can be a score.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary flow diagram of an exemplary convolutional neural network according to an exemplary embodiment of the present disclosure;

FIG. 2 is a combined exemplary schematic/flow diagram of an exemplary neural network architecture for the exemplary convolutional neural network according to an exemplary embodiment of the present disclosure;

FIGS. 3A-3D are exemplary pixel-wise heat maps generated using the exemplary system, method, and computer-accessible medium according to an exemplary embodiment of the present disclosure;

FIG. 4 is an exemplary flow diagram of a method for determining a risk of developing breast cancer for a patient according to an exemplary embodiment of the present disclosure; and

FIG. 5 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary system, method, and computer-accessible medium, according to an exemplary embodiment of the present disclosure, can include the determination of breast cancer risk using various exemplary imaging modalities. For example, the exemplary system, method, and computer-accessible medium is described below using mammographic images. However, the exemplary system, method, and computer-accessible medium can also be used on other suitable imaging modalities, including, but not limited to, magnetic resonance imaging, positron emission tomography, ultrasound, and computed tomography.

A case-control study was performed retrospectively utilizing a screening mammogram database. Average risk screening women were evaluated by excluding women who have personal history of breast cancer, family history of breast cancer, and any known genetic mutation that increases the risk for breast cancer. After applying the exclusion criteria, 210 patients were identified consecutively with a new first time diagnosis of breast cancer. Mammograms from these patients, at least 2 years (e.g., median 3.3 years, range 2.0-5.3 years) prior to developing breast cancer, were identified and made up the “high risk” case group composed of the bilateral craniocaudal mammographic dataset (e.g., 420 total). The control group consisted of 527 patients without breast cancer from the same time period. Prior mammograms from these patients made up the “low risk” control group composed of the bilateral craniocaudal mammographic data-set (e.g., 1054 total). These 527 patients in the control group had documented negative follow-up mammogram for at least 2 years (e.g., median 3.1 years, range 2.0-4.8 years).

From each patient, the age and the BI-RADS mammographic density assessment was recorded on a 4-point scale (e.g., 1-fatty, 2-scattered, 3-heterogeneously dense, and 4-extremely dense) by one of five breast fellowship trained radiologists. Mammograms were performed on dedicated mammography units (e.g., Senographe Essential, GE Healthcare). Of patients who developed breast cancer, histologic subtype was recorded based on the World Health Organization classification. (See, e.g., Reference 13). Statistical analysis was performed using the IBM SPSS software (version 24).

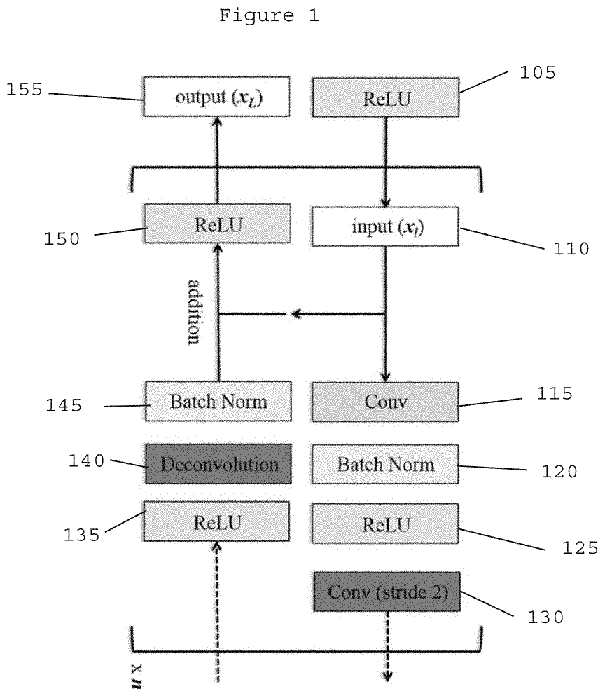

FIG. 1 shows an exemplary flow diagram of the exemplary CNN according to an exemplary embodiment of the present disclosure. For example, as shown in FIG. 1, a rectified linear unit (“ReLu”) layer 105 can be input 110 to one or more convolutional layers 115. Batch normalization 120 can be performed prior to a further ReLu layer 125. A convolutional kernel 130 having a stride length of 2 can be included in the exemplary CNN. In order to produce an output 155, a ReLu layer 135 can be deconvolved at procedure 140. A further batch normalization 145 can be performed, the output of which can be combined with input 110 into ReLu layer 150 to produce output 155.

As shown in the exemplary flow diagram of FIG. 1, the exemplary fully CNN can be implemented using a series of upsampling convolutional transpose operators performed on the deepest network layers, resulting in a dense classification matrix equal in dimension to the original image size for each forward pass. (See, e.g., Reference 14). Asymmetric contracting and expanding topology that efficiently combines low- and high-level features can be used. (See, e.g., Reference 15). Concatenation operations can be replaced with residual connections, and associated projection matrices to match feature layer dimensions. The exemplary system, method, and computer-accessible medium can use residual neural networks to stabilize gradients during backpropagation, resulting in improved optimization and facilitating greater network depth. Furthermore in asymmetric contracting and expanding topology, residual connections can facilitate the network to learn the appropriate feature depth, as contributions from the deepest, large field-of-view feature maps can be selectively eliminated through identity mappings.

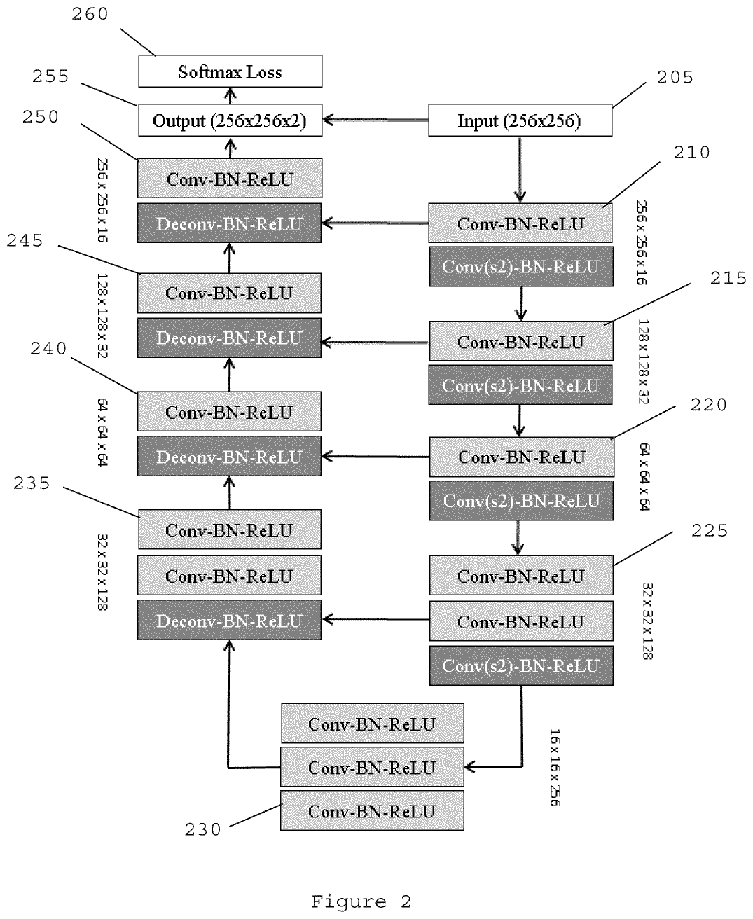

FIG. 2 shows a combined exemplary schematic/flow diagram of the exemplary neural network architecture for the exemplary CNN according to an exemplary embodiment of the present disclosure. As shown in FIG. 2, an input image 205 (e.g., of a size of 256×256 pixels) can be input into a plurality of layers (e.g., layer 210, layer 215, layer 220, and layer 225) having different sizes. The exemplary CNN can be implemented by series of 3×3 convolutional kernels (e.g., kernels 230) to prevent overfitting. (See, e.g., References 17-20). The exemplary system, method, and computer-accessible medium, according to an exemplary embodiment of the present disclosure, can implement the exemplary procedures with or without pooling layers. For example, if no pooling layers are utilized, downsampling can be implemented using a 3×3 convolutional kernel with stride length of 2 to decrease the feature maps by 75% in size. All non-linear functions can be modeled by the ReLu. (See, e.g., References 17-20). Batch normalization can be used between the convolutional and ReLu layers to limit drift of layer activations during training. (See, e.g., Reference 21). In successively deeper layers, the number of feature channels can gradually increase from 16, 32, 64, 128, and 256, reflecting increasing representational complexity.

As shown in FIG. 2, the exemplary contracting and expanding fully CNN can be composed entirely of 3×3 convolutions, a total of four strided convolutions (e.g., layers 210, 215, 220, and 225) and convolutional transpose operations are incorporated, instead of pooling layers and symmetric residual connections. Each mammogram can be normalized as a map of z-scores and resized to an input image size of 256×256. Data augmentation can include real-time modifications to the source images at the time of training. Specifically, 50% of all images in a mini-batch were modified randomly using: (i) addition across all pixels of a scalar between [−0.1, 0.1]; and (ii) random affine transformation of the original mammogram. Given a two-dimensional affine matrix,

$$[ \begin{matrix}{s}_{1} & {t}_{1} & {r}_{1} \\ {t}_{2} & {s}_{2} & {r}_{2} \\ 0 & 0 & 1\end{matrix} ]$$

the random affine transformation was initialized with random uniform distributions of interval s₁,s₂ ϵ[0.8, 1.2], t₁,t₂ ϵ[−0.03, 0.3] and r₁, r₂ ϵ[−128, 128]. Four increasing layers (e.g., layer 235, layer 240, layer 245, and layer 250, can be used to produce an output 255, and a final softmax score 260 that can be used for risk classification. A softmax score of about 0.5 or above (e.g., above 0.45) can indicate a high risk of developing breast cancer.

Training was implemented using the Adam optimizer, and a procedure for first-order gradient-based optimization of stochastic objective functions, based on adaptive estimates of lower-order moments. (See, e.g., References 17-20). Parameters can be initialized using an exemplary heuristic. (See, e.g., Reference 16). L2 regularization can be implemented to prevent over-fitting of data by limiting the squared magnitude of the kernel weights. To account for training dynamics, the learning rate can be annealed and the mini-batch size can be increased whenever training lost plateaus. Furthermore, a normalized gradient procedure can be utilized to facilitate locally adaptive learning rates that adjust according to changes in the input signal. (See, e.g., References 17-20). The overall training time was 6 hours.

For statistical analysis, cases were separated into 80% training (e.g., 590/737) and 20% test sets (e.g., 147/737). A 5-fold cross validation was performed. A final softmax score threshold of 0.5 from the average of raw logits from each pixel was used for two class classification.

### Exemplary Results

The average age of patients between the case and the control groups was not statistically different [case: 57.4 years (SD, 10.4) and control: 58.2 years (SD, 10.9), p=0.33]. All 210 patients had unilateral breast cancers; 69.5% (e.g., 146/210) had invasive ductal carcinoma; 19% (e.g., 40/210) had ductal carcinoma in situ; 7.1% (e.g., 15/210) had invasive lobular carcinoma; 4.3% (e.g., 9/210) had mixed lobular and ductal invasive carcinoma, and 17.6% (e.g., 37/210) of the patients had multifocal disease.

Breast Density (“BD”) was significantly higher in the case group [2.39 (SD, 0.7)] than the control group [1.98 (SD, 0.75), p<0.0001]. Using multivariate logistic regression analysis, both CNN pixel-wise mammographic risk model and BD were significant independent predictors of breast cancer risk (e.g., p<0.0001). The CNN risk model showed greater predictive potential [OR=4.42 (95% CI, 3.4-5.7] compared to BD [OR=1.67 (95% CI, 1.4-1.9).

There was a strong signification correlation of CNN pixel-wise mammographic risk results between the left and right breast (e.g., Pearson correlation, r=0.90, n=737). In the case group, there was a significant correlation between the left and right breast (e.g., Pearson correlation, r=0.86, n=210). In the control group, there was a signification correlation between the left and right breast (e.g., Pearson correlation, r=0.86, n=527).

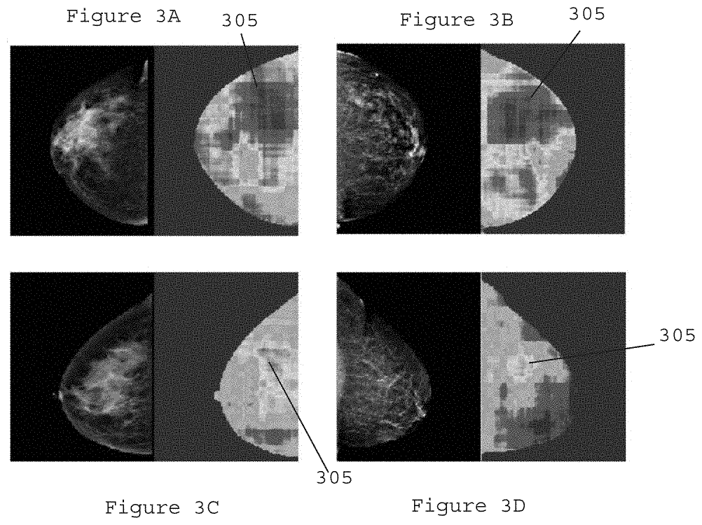

The CNN risk model achieved an overall accuracy of 72% (e.g., 95% CI, 69.8-74.4%) in predicting patients in the case group. FIGS. 3A-3D show exemplary pixel-wise heat maps generated using the exemplary system, method, and computer-accessible medium according to an exemplary embodiment of the present disclosure. Heat maps were generated on a pixel-wise basis, showing subregions within the mammogram that are most commonly encountered in normal (e.g., FIGS. 3C and 3D) and high cancer risk (e.g., FIGS. 3A and 3B) patients. Mammogram images in FIGS. 3A and 3B shows similar breast densities (e.g., heterogeneously dense) and mammograms in FIGS. 3C and 3D illustrate similar breast densities (e.g., scattered) but the corresponding heat maps are different with patient A with significantly higher mammographic regions containing red and correctly identifying high risk. Similarly, patient C with significantly higher mammographic regions containing high risk areas 305 and correctly identifying high risk.

The exemplary CNN was trained for a total of 144,000 iterations (e.g., approximately 1170 epochs with a batch size of 12) before convergence. A single forward pass through during test time for classification of new cases can be achieved in 0.063 seconds.

### Exemplary Discussion

The exemplary system, method, and computer-accessible medium, according to an exemplary embodiment of the present disclosure, can include pixel-wise cancer risk assessment using mammogram to define risk on an individual basis. For example, an overall accuracy of 72% was achieved in predicting high versus low cancer risk mammograms.

With changing screening guidelines, it can be beneficial to better define an individual's risk for breast cancer. While mammographic breast density categorization procedures exist, accurate identification of who can be at high risk remains a challenge. In contrast, the exemplary system, method, and computer-accessible medium, which can utilize heat maps, can provide and show a breast cancer risk heterogeneity among mammographic breast density categories. For example, not all heterogeneously dense breasts are high risk, with a subset demonstrating a stronger resemblance to a low risk pattern. Similarly, not all breasts with the scattered fibroglandular density demonstrate a low risk pattern. While approximately half the population can be categorized as having dense breasts (e.g., BIRADS 3 and 4) (see, e.g., Reference 11), the exemplary system, method, and computer-accessible medium, according to an exemplary embodiment of the present disclosure, can be used to better classify low and high risk patients.

The exemplary CNN, according to an exemplary embodiment of the present disclosure, did not show any significant bias toward the cancer side. In addition, significant correlation was observed between the two breasts (e.g., the side that developed cancer and the contralateral non-cancer side), indicating that the exemplary CNN can predict risk for breast cancer based on features that are largely conserved on an individual basis. In particular, the heat maps shown in FIGS. 3A-3D show regions within the breast that have the most overlapping mammographic features with patients who subsequently developed cancer. The overlapping features come from both breasts (e.g., the side that developed cancer and the contralateral side that never developed cancer).

Individualized breast cancer risk stratification can significantly impact clinical management. This risk assessment could be implemented into screening guidelines. In the setting of later and less frequent evolving screening guidelines for average risk women, accurately categorized high risk women can benefit from earlier and more frequent screening.

Beyond screening, individualized risk assessment can be used for chemoprevention strategies. The American Society of Clinical Oncology, National Comprehensive Cancer Network, and United States Preventive Services Task Force recommend counseling high risk women above the age of 35 on pharmacologic interventions for breast cancer risk reduction. (See, e.g., References 26-28). Two selective estrogen receptor modulators, tamoxifen and raloxifene, approved for chemoprevention in the United States, show up to a 50% cancer risk reduction. Additionally, two aromatase inhibitors, exemestane and anastrozole, not yet approved for use in the United States, have shown significant chemopreventive potential in preliminary studies. (See, e.g., Reference 29). Individualized breast cancer risk assessment can have potential to aid in the selection of high risk patients and counseling on chemoprevention.

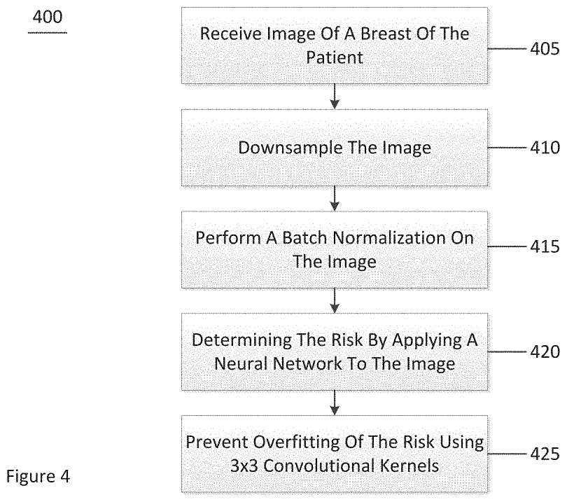

FIG. 4 shows an exemplary flow diagram of a method 400 for determining a risk of developing breast cancer for a patient according to an exemplary embodiment of the present disclosure. For example at procedure 405, an image of a breast of a patient can be received. At procedure 410, the image can be downsampled. At procedure 415, a batch normalization can be performed on the image. At procedure 420, a risk of developing breast cancer can be determined by applying a neural network (e.g., a CNN). At procedure 425, overfitting of the risk determination can be prevented (e.g., using 3×3 convolutional kernels).

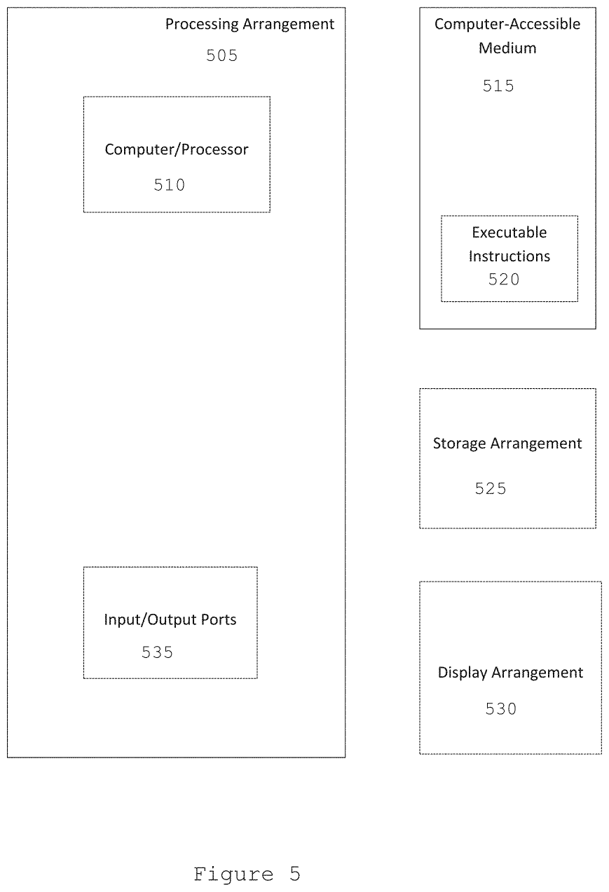

FIG. 5 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 505. Such processing/computing arrangement 505 can be, for example entirely or a part of, or include, but not limited to, a computer/processor 510 that can include, for example one or more microprocessors, and use instructions stored on a computer-accessible medium (e.g., RAM, ROM, hard drive, or other storage device).

As shown in FIG. 5, for example a computer-accessible medium 515 (e.g., as described herein above, a storage device such as a hard disk, floppy disk, memory stick, CD-ROM, RAM, ROM, etc., or a collection thereof) can be provided (e.g., in communication with the processing arrangement 505). The computer-accessible medium 515 can contain executable instructions 520 thereon. In addition or alternatively, a storage arrangement 525 can be provided separately from the computer-accessible medium 515, which can provide the instructions to the processing arrangement 505 so as to configure the processing arrangement to execute certain exemplary procedures, processes, and methods, as described herein above, for example.

Further, the exemplary processing arrangement 505 can be provided with or include an input/output ports 535, which can include, for example a wired network, a wireless network, the internet, an intranet, a data collection probe, a sensor, etc. As shown in FIG. 5, the exemplary processing arrangement 505 can be in communication with an exemplary display arrangement 530, which, according to certain exemplary embodiments of the present disclosure, can be a touch-screen configured for inputting information to the processing arrangement in addition to outputting information from the processing arrangement, for example. Further, the exemplary display arrangement 530 and/or a storage arrangement 525 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties:
1. Siegel R L, Miller K D, Jemal A. Cancer statistics, 2017. C A Cancer J Clin 2017; 67:7-30.
2. Madigan M P, Ziegler R G, Benichou J, et al. Proportion of breast cancer cases in the United States explained by well-established risk factors. J Natl Cancer Inst 1995; 87:1681-1685
3. Freer P E. Mammographic breast density: impact on breast cancer risk and implications for screening. Radiographics 2015; 35:302-315.
4. Wolfe J N. Breast patterns as an index of risk for developing breast cancer. Am J Roentgenol 1976; 126:1130-1137.
5. Boyd N F, Byng J W, Jong R A, et al. Quantitative classification of mammographic densities and breast cancer risk: results from the Canadian National Breast Screening Study. J Natl Cancer Inst 1995; 87:670-675.
6. Gram I T, Funkhouser E, Tabar L. The Tabar classification of mammographic parenchymal patterns. EurJ Radiol 1997; 24:131-136.
7. Vacek P M, Geller B M. A prospective study of breast cancer risk using routine mammographic breast density measurements. Cancer Epidemiol Biomark Prev 2004; 13:715-722.
8. Kerlikowske K, Ichikawa L, Miglioretti D L, et al. Longitudinal measurement of clinical mammographic breast density to improve estimation of breast cancer risk. J Natl Cancer Inst 2007; 99:386-395.
9. Siu A L. U.S. Preventive Services Task Force. Screening for breast cancer: U.S. Preventive Services Task Force recommendation statement. Ann Intern Med 2016; 164:279-296.
10. Oeffinger K C, Fontham E T H, Etzioni R, et al. Breast cancer screening for women at average risk: 2015 guideline update from the American Cancer Society. JAMA. 2015; 314:1599-1614.
11. Kerlikowske K, Zhu W, Hubbard R A, et al. Outcomes of screening mammography by frequency, breast density, and postmenopausal hormone therapy. JAMA Intern Med 2013; 173:807-816.
12. LeChun Y, Bengio T, Hinton G. Deep learning. Nature 2015; 521:436-444.
13. American Joint Committee on Cancer. AJCC Cancer Staging Manual. 7th ed. New York, N Y: Springer, 2010.
14. Long J, Shelhamer E, Darrell T. Fully convolutional networks for semantic segmentation. In: In: Proceedings of the IEEE Conference on Computer Vision and Pattern Recognition; 2015.
15. Ronneberger O, Fischer P, Brox T. U-net: convolutional networks for biomedical image segmentation. In: International Conference on Medical Image Computing and Computer-Assisted Intervention. Springer; 2015.
16. He K, Zhang X, Ren S, et al. Delving deep into rectifiers: surpassing human-level performance on ImageNet classification. Comput Vision Pattern Recognit arXiv: 1502.01852
17. Simonyan K, Zisserman A. Very deep convolutional networks for large-scale image recognition. Int Conf Learning Represent 2015: 1-14.
18. Nair V, Hinton G E. Rectified linear units improve restricted Boltzmann machines. In: In: Proceedings of the 27th International Conference on Machine Learning, Haifa, Israel; 2010.
19. Srivastava N, Hinton G E, Krizhevsky A, et al. Dropout: a simple way to prevent neural networks from overfitting. J Mach Learn Res 2014; 15:1929-1958.
20. Kingma D P, Ba J. Adam: a method for stochastic optimization. Machine Learning arXiv:1412.6980.
21. Ioffe S, Szegedy C. Batch normalization: accelerating deep network training by reducing internal covariate shift. In: International Conference on Machine Learning; 2015.
22. Wei J, Chan H P, Wu Y T, et al. Association of computerized mammographic parenchymal pattern measure with breast cancer risk: a pilot case-control study. Radiology 2011; 260:42-49.
23. Heidari M, Khuzani A Z, Hollingsworth A B, et al. Prediction of breast cancer risk using a machine learning approach embedded with a locality preserving projection algorithm. Phys Med Biol 2018; 63:035020.
24. Tan M, Pu J, Cheng S, et al. Assessment of a four-view mammographic image feature based fusion model to predict near-term breast cancer risk. Ann Biomed Eng 2015; 43:2416-2428.
25. Li Y, Fan M, Cheng H, et al. Assessment of global and local region-based bilateral mammographic feature asymmetry to predict short-term breast cancer risk. Phys Med Biol 2018; 63:025004.
26. Visvanathan K, Hurley P, Bantug E, et al. Use of pharmacologic interventions for breast cancer risk reduction: American Society of Clinical Oncology clinical practice guideline. J Clin Oncol 2013; 31:2942-2962.
27. National Comprehensive Cancer Network. The NCCN Clinical Practice Guidelines in Oncology (NCCN Guidelines) Breast Cancer Risk Reduction (version 1.2014). www.NCCN.org; 2014.
28. Moyer V A. Medications to decrease the risk for breast cancer in women: recommendations from the U.S. Preventive Services Task Force recommendation statement. Ann Intern Med 2013; 159:698-708.
29. Pruthi A, Heisey R E, Bevers T B. Chemoprevention for breast cancer. Ann Surg Oncol 2015; 22:3230-3235.
30. Ciatto S, Houssami N, Apruzzese A, et al. Categorizing breast mammographic density: intra- and interobserver variability of B I-RADS density categories. Breast 2005; 14:269-275.
31. Ciatto S, Houssami N, Apruzzese A, et al. Reader variability in reporting breast imaging according to B I-RADS assessment categories (the Florence experience). Breast 2006; 15:44-51.
32. Ooms E A, Zonderland H M, Eijkemans M J, et al. Mammography: interobserver variability in breast density assessment. Breast 2007; 16:568-576.

What is claimed is:

1. A non-transitory computer-accessible medium having stored thereon computer-executable instructions for determining a risk of developing breast cancer for at least one patient, wherein, when a computer arrangement executes the instructions, the computer arrangement is configured to perform procedures comprising:
receiving at least one image of at least one internal portion of a breast of the at least one patient; and
determining the risk by applying at least one neural network to the at least one image.

2. The computer-accessible medium of claim 1, wherein the neural network is a convolutional neural network (CNN).

3. The computer-accessible medium of claim 2, wherein the CNN includes a plurality of layers.

4. The computer-accessible medium of claim 3, wherein each of the layers has a different number of feature channels.

5. The computer-accessible medium of claim 3, wherein the CNN includes at least four layers.

6. The computer-accessible medium of claim 5, wherein (i) a first layer of the at least four layers has 256×256×16 feature channels, (ii) a second layer of the at least four layers has 128×128×32 feature channels, (iii) a third layer of the at least four layers has 64×64×64 feature channels, and (iv) a fourth layer of the at least four layers has 32×32×128 feature channels.

7. The computer-accessible medium of claim 2, wherein the CNN includes 3×3 convolutional kernels.

8. The computer-accessible medium of claim 7, wherein the computer arrangement is further configured to prevent overfitting of the risk using the 3×3 convolutional kernels.

9. The computer-accessible medium of claim 2, wherein the CNN excludes pooling layers.

10. The computer-accessible medium of claim 2, wherein the computer arrangement is further configured to downsample the at least one image.

11. The computer-accessible medium of claim 10, wherein the computer arrangement is configured to downsample the at least one image using a 3×3 convolutional kernel.

12. The computer-accessible medium of claim 11, wherein the 3×3 convolutional kernel has a stride length of 2.

13. The computer-accessible medium of claim 2, wherein the computer arrangement is configured to determine the risk by modeling non-linear functions using at least one rectified linear unit (“ReLu”) layer.

14. The computer-accessible medium of claim 13, wherein the computer arrangement is further configured to perform a batch normalization on the at least one image.

15. The computer-accessible medium of claim 14, wherein the computer arrangement is configured to perform the batch normalization to limit drift of layer activations.

16. The computer-accessible medium of claim 14, wherein the computer arrangement is configured to perform the batch normalization between the at least one ReLu layer and a convolutional layer.

17. The computer-accessible medium of claim 2, wherein the CNN includes four strided convolutions.

18. The computer-accessible medium of claim 1, wherein the at least one risk is a score.

19. A method for determining a risk of developing breast cancer for at least one patient, comprising:
receiving at least one image of at least one internal portion of a breast of the at least one patient; and
using a computer arrangement, determining the risk by applying at least one neural network to the at least one image.

20. A system for determining a risk of developing breast cancer for at least one patient, comprising:
a computer hardware arrangement configured to:
receive at least one image of at least one internal portion of a breast of the at least one patient; and
determine the risk by applying at least one neural network to the at least one image.
